(19) United States
(12) Patent Application Publication
(10) Pub. No.: US 2008/0020474 A1
(43) Pub. Date: Jan. 24, 2008

(54) Method of Analyzing Biosample by Laser Ablation and Apparatus Therefor

(75) Inventors: Yoshihide Hayashizaki, Ibaraki (JP); Jun Kawai, Kanagawa (JP); Toshizo Hayashi, Tokyo (JP); Mamoru Kamiya, Tokyo (JP)

Correspondence Address: BIRCH STEWART KOLASCH & BIRCH, PO BOX 747, FALLS CHURCH, VA, 22040-0747, US

(73) Assignees: RIKEN, Wako-Shi, Saitama (JP); Kabushiki Kaisha Dnaform, Tokyo (JP)

(21) Appl. No.: 10/594,770

(22) PCT Filed: Mar. 29, 2005
(86) PCT No.: PCT/JP05/05809

§ 371 Date: Jun. 28, 2007

(30) Foreign Application Priority Data
Mar. 30, 2004 (JP) 2004-097260

(51) Int. Cl. G01N 33/48; B01L 11/00; G01N 1/00; G01N 24/00

(52) U.S. Cl. 436/86; 422/99; 436/173; 436/174

(57) ABSTRACT

A method of analyzing a biosample that enables substantial shortening of time required for analysis, further enabling obtaining highly reliable results through means for avoiding sway of analytical results depending on observers, and that enables one-time analysis of a multiplicity of genes, etc. on a single biosample to thereby enhance workload and time efficiencies, and that enables analysis of multiple genes, etc. under conditions completely free from any difference in background attributed to biosamples. There is provided a method comprising irradiating a biosample as an analyte with ultra-short pulse laser beams to thereby effect an ablation thereof so that molecules contained in the biosample are atomized into constituting elements, ionizing the constituting elements resulting from the atomization and analyzing the ionized constituting elements to thereby analyze analytical-target molecules of the biosample.

### TECHNICAL FIELD

The present invention relates to a method of analyzing a biosample using laser ablation and an apparatus therefor, more particularly to a method of analyzing a biosample using laser ablation and an apparatus therefor, which are capable of significantly improving analysis efficiency comparing to a conventional ones. For example, the invention relates to a method of analyzing a biosample using laser ablation and an apparatus therefor, in which a biotissue section is used as the biosample and which are preferable for use in the mass spectrometry of nucleic acid in the biotissue section.

### BACKGROUND ART

Conventionally, as a method of analyzing a biosample such as a biotissue section, various chemical staining method and methods utilizing the hybridization of a nucleic-acid probe (in situ hybridization method) have mainly been used.

Herein, the chemical staining method is a method of observing a tissue, the morphology of a cell, and the existence and the localization of a substance to be analyzed under a microscope by utilizing the fact that the way of staining is different depending on a type of substance contained in a tissue.

On the other hand, the in situ hybridization method is a method where a labeled nucleic-acid probe is hybridized with nucleic acid that exists in a tissue to make it possible to detect mRNA being transferred in a cell or observe the transcription activity of a gene.

Further, in the analysis of a biosample such as a biotissue section, analysis of a tissue section by using a labeled antibody is frequently used similar to the above-described method.

However, in the above-described conventional various method of analyzing biosample, there existed a problem that processes required for staining and color development was complicated and time required in analysis became long time.

Further, the above-described conventional various methods of analyzing a biosample, a man needs to analyze the biotissue section or the like after processing by observing it under a microscope, there was a problem that a qualitative analysis was mainly done and different results were obtained by observing a same section due to the skill level and the subjective of observers.

Furthermore, the above-described conventional various methods of analyzing a biosample, only two types of genes can be measured at one time on a same biotissue section, there existed a problem that it was difficult to perform analysis of a plurality of genes under conditions completely free from any difference in background attributed to biotissue sections.

It is to be noted that prior art that the applicant of this application knows at the point of filing for patent is as described above but not inventions in reference publicly known invention, so that there is no prior art information that should be described.

### DISCLOSURE OF THE INVENTION

### Problems to be Solved By the Invention

The present invention has been created in view of the above-described various problems that the prior art has, and it is an object of the invention to provide a method of analyzing a biosample using laser ablation and an apparatus therefor, which enables significant shortening of time required for analysis comparing to a conventional art.

Further, it is an object of the present invention to provide a method of analyzing a biosample using laser ablation and an apparatus therefor, which enables the obtaining of highly reliable results avoiding sway of analytical result depending on observers.

Further, it is an object of the present invention to provide a method of analyzing a biosample using laser ablation and an apparatus therefor, which enables one-time analysis of a multiplicity of genes on a single biosample to enhance workload and time efficiency, and also enables analysis of a plurality of genes under conditions completely free from any difference in background attributed to biosamples.

Further, it is an object of the present invention to provide a method of analyzing a biosample using laser ablation and an apparatus therefor, which eliminates fear that the analysis of mass spectrum becomes difficult, and high resolving power is not required for a mass spectrometer in the case of performing the mass spectrometry of molecules in a biosample.

Further, it is an object of the present invention to provide a method of analyzing a biosample using laser ablation and an apparatus therefor, which enables realizing of the atomization and the ionization of constituting atoms constituting the molecules in the biosample simultaneously by one laser source and to enables significant simplification of laser irradiation control.

Further, it is an object of the present invention to provide a method of analyzing a biosample using laser ablation and an apparatus therefor, which enables efficient analysis even in the state where many types of labeled isotopes are mixed.

### Means for Solving the Problems

To achieve the above-described objects, the method of analyzing a biosample using laser ablation and the apparatus therefor according to the present invention are that molecules in a biosample are atomic ionized to produce atomic ions by ablating the biosample by ultra-short pulse laser beams, and the produced atomic ions are analyzed. Thus, it is possible to conduct chemical analysis of the molecules in the biosample.

Specifically, in the method of analyzing a biosample using laser ablation and the apparatus therefor according to the present invention, the molecules in the biosample are decomposed into pieces and atomized for each atom constituting the molecules by ablating the biosample by the ultra-short pulse laser beams, and at the same time, the atomized atoms are ionized into univalent ions, and quantitative analysis is made possible by analyzing the atomic ions produced by the ionization.

Therefore, by the method of analyzing a biosample using laser ablation and the apparatus therefor according to the present invention, processes until a nucleic-acid probe is hybridized (process of about 2 hours), for example, are the same as a conventional in situ hybridization, but time (about 24 hours) spent in the subsequent color development and sensitization is not needed and it can be directly set in a spectrometer, so that time required for analysis can be significantly shortened.

Further, according to the method of analyzing a biosample using laser ablation and the apparatus therefor according to the present invention, an analytical result is produced as quantitative data, so that it does not have scattering caused by observers and highly reliable result can be obtained.

Furthermore, only 2 to 3 types of genes can be measured at one time in a same biosample in the above-described method by the conventional art, but according to the method of analyzing a biosample using laser ablation and the apparatus therefor of the present invention, a multiplicity of genes or the like can be measured at one time on a single biosample because there are so many types of genes that can be used as a label, so that workload and time efficiencies can be improved. In addition, analysis can be performed on a single biosample, so that analysis of a plurality of genes or the like can be performed under conditions completely free from any difference in background attributed from the biosample.

Further, in the case of performing mass spectrometry by the method of analyzing a biosample using laser ablation and the apparatus therefor according to the present invention, mass spectrometry is performed to atomic ions having low mass, so that it not only eliminates the fear that the analysis of mass spectrum becomes difficult, but also mass spectrometer having high resolving power does need to be used.

Further, according to the method of analyzing a biosample using laser ablation and the apparatus therefor according to the present invention, by ablating the biosample by one type of ultra-short pulse laser beams, the atomization of the molecules in the biosample and the ionization of the atomized atoms into monovalent ion can be simultaneously performed efficiently. Therefore, laser irradiation control is simplified, and for example, many types of labeled elements can be simultaneously used in performing chemical analysis, so that analysis efficiency can be remarkably improved.

In other words, in the method of analyzing a biosample using laser ablation and the apparatus therefor according to the present invention, the atomization and the ionization of the labeled elements can be performed simultaneously by one type of the ultra-short pulse laser beams, so that an analysis operation can be simplified significantly and efficiency of analysis can be remarkably improved comparing to the conventional methods.

Furthermore, since the above-described ionization is ionization (non-resonant ionization) that is performed through a non-resonant process using the high peak value intensity of the ultra-short pulse laser beams, each labeled atom can be severally ionized even in the state where many types of labeled isotopes are mixed, by which application to a multi-label system is easy, and highly accurate and highly efficient analysis of polymer can be performed.

Specifically, the present invention is that by irradiating the ultra-short pulse laser beams onto the biosample to be analyzed to perform ablation, molecules contained in the above-described biosample are atomized into constituting elements, the above-described atomized constituting elements are ionized, and by analyzing the above-described ionized constituting elements, analytical-target molecules in the above-described biosample are thus analyzed.

Further, the present invention is that by directly or indirectly labeling a substance having specific bond to the analytical-target molecules in the above-described biosample, and by analyzing molecules to which the above-described labeled substance is bonded, the analytical-target molecules in the above-described biosample are thus analyzed.

Further, the present invention is that the above-described labeled substance having specific bond is nucleic acid.

Further, the present invention is that the analytical-target molecules in the above-described biosample are nucleic acid.

Further, the present invention is that the nucleic acid being the above-described labeled substance having specific bond contains DNA, RNA, PNA or other modified nucleic acid.

Further, the present invention is that the above-described labeled substance having specific bond, which is used for analyzing the above-described nucleic acid, is bonded by hybridization.

Further, the present invention is that the above-described labeled substance having specific bond, which is used for analyzing the above-described nucleic acid, is aptamer.

Further, the present invention is that the above-described labeling of nucleic acid for analyzing the above-described nucleic acid is conducted by a TUNEL method.

Further, the present invention is that the above-described analytical-target molecules in the biosample are protein.

Further, the present invention is that the above-described labeled substance having specific bond, which is used for analyzing the above-described protein, is bonded by antigen-antibody reaction.

Further, the present invention is that the above-described label is an element label.

Further, the present invention is that the above-described element label is a stable element isotopic label.

Further, the present invention is that analysis of the above-described ionized constituting element is mass spectrometry.

Further, the present invention is that the above-described mass spectrometry is mass spectrometry by a time-of-flight method.

Further, the present invention is that multi-channeling is conducted by using plural types of labels as a label, and at least 2 types or more molecules in a single biosample are analyzed as analytical-target molecules.

Further, the present invention is that by allowing an tissue image obtained by observing the above-described biosample by a microscope to correspond to the position of the above-described ablated spot, the localization of the analytical-target molecules in the above-described biosample is analyzed.

Further, the present invention is that the above-described ultra-short pulse laser beams have a pulse time width of 1 femto second or more and 1 pico second or less, and a peak value output of 1 mega watt or more and 10 giga watts or less.

Further, the present invention is that the above-described biosample is a biotissue section or a smear sample.

Further, the present invention has: an ultra-short pulse laser generating unit capable of outputting ultra-short pulse laser beams, which atomizes molecules contained in the above-described biosample into constituting elements by irradiating the beams onto a biosample to be analyzed and ablating the biosample, and ionizes the above-described atomized constituting elements; an analyzer that introduces and analyzes the constituting elements ionized by the ultra-short pulse laser beams that are outputted from the above-described ultra-short pulse laser generating unit; and a microscope unit for observing the shape of the above-described biosample to be analyzed.

Further, the present invention is that the above-described microscope unit is an upright microscope unit, the objective lens of the above-described upright microscope unit is arranged on the upper surface the above-described biosample, and the irradiation of the ultra-short pulse laser beams from the above-described ultra-short pulse laser generating unit is performed from the lower surface of the above-described biosample.

Further, the present invention is that the above-described microscope unit is the upright microscope, the objective lens of the above-described upright microscope is arranged on the upper surface of the above-described biosample, and the irradiation of ultra-short pulse laser beams from the above-described ultra-short pulse laser generating unit is performed from the upper surface the above-described biosample.

Further, the present invention is that the above-described microscope unit is an inverted microscope, the objective lens of the above-described inverted microscope is arranged on the lower surface of the above-described biosample, and the irradiation of ultra-short pulse laser beams from the above-described ultra-short pulse laser generating unit is performed from the upper surface the above-described biosample.

Further, the present invention is that the above-described microscope unit is an inverted microscope, the objective lens of the above-described inverted microscope is arranged on the lower surface of the above-described biosample, and the irradiation of ultra-short pulse laser beams from the above-described ultra-short pulse laser generating unit is performed from the lower surface the above-described biosample.

Further, the present invention is that the above-described ultra-short pulse laser generating unit is allowed to output ultra-short pulse laser beams whose pulse time width is 1 femto second or more and 1 pico second or less, and whose peak value output is 1 mega watt or more and 10 giga watts or less.

Further, the present invention further has an image analysis apparatus that analyzes images observed by the above-described microscope unit.

Further, the present invention is that the above-described biosample is a biotissue section or a smear sample.

Herein, in ablating the biosample by the ultra-short pulse laser beams in the present invention, irradiation of the ultra-short pulse laser beams on the biosample by 1 shot (1 pulse) is enough. However, the ultra-short pulse laser beams may be irradiated by a plurality of shots (plural pulses) on the biosample, and the shot number (pulse number) ultra-short pulse laser beams to be irradiated may be appropriately selected.

Further, it is preferable that the ultra-short pulse laser beams have the pulse time width of 10 pico seconds or less, and it is appropriate to use a femto-second laser beam that is irradiated from a laser that is usually called a femto-second laser having the pulse time width of 1 femto second or more and 1 pico second or less, for example.

Further, it is preferable that the peak value output of the ultra-short pulse laser beams be 1 mega watt or more, and more particularly, 1 mega watt or more and 10 giga watts or less is more preferable.

It is because there is a fear that multivalent ions are produced and analysis of mass spectrum becomes difficult when the peak value output of the ultra-short pulse laser beams are larger than 10 giga watts, and the efficiency of atomization/ionization reduces to make it difficult to observe atomic ion signal when the peak value output of the ultra-short pulse laser beams are smaller than 1 mega watt.

Meanwhile, according to the experiment conducted by the inventor of this invention (described later), excellent result could be obtained when the pulse time width was 100 femto seconds with the laser power of 0.2 mJ.

Further, according to the present invention, the ultra-short pulse laser beams such as the femto second laser beam capable of efficiently performing ionization simultaneously with atomization is irradiated on nucleic acid labeled by an isotopic element or the like. Therefore, there is no need to selectively ionize the labeled elements and various types of labeled elements can be used. In addition, since the repetition rate of laser irradiation can be increased to several kHz, it is suitable for high-speed analysis.

Further, in the present invention, by moving at least one of the ultra-short pulse laser beams that ablate the molecules in the biosample and the biosample to be analyzed, the short pulse laser beams are allowed to ablate and analyze the biosample to be analyzed without omission and duplication. Specifically, in the present invention, ablation of biosample over a wide area without omission and duplication is made possible by the relative movement of a spot of the ultra-short pulse laser beams and the biosample to be analyzed as a sample, for example.

According to the present invention, analysis speed becomes significantly faster than a conventional one due to these characteristics.

Further, according to the present invention, the use of element labels as a label is made possible. More particularly, the use of many types of isotopic elements as the element label is made possible, and the types of labels can be increased to the number (270 types) of many types of stable isotopes when stable isotopic elements, for example, are used as the element label. This means that amount of information can be increased dramatically comparing to a fluorescence method (2 to 6 types) being a conventional labeling method or radioactive isotopic elements (about 10 types).

More particularly, as a label of analytical-target molecules in the biosample, it becomes possible to use group 1 stable isotope in the periodic table such as ³⁹K and ⁴¹K, group 16 stable isotope in the periodic table such as ³²S and ³⁵S, group 17 stable isotope in the periodic table such as ³⁵Cl and ³⁷Cl, the stable isotope of transition metal in the periodic table such as ¹¹⁸Sn and ¹²⁰Sn, furthermore, group 8 Fe in the periodic table, group 12 Hg in the periodic table, and a stable isotopic element of lanthanoid, which is group 15 in the periodic table, such as I, Eu, Tb, Sm and Dy, for example.

Herein, comparing to the labels currently used, the variety of labels can be increased to as many as 270 types if the stable isotopic elements, for example, are used.

Furthermore, when multi-channeled by using a plurality of labels as labels, at least 2 types or more of molecules in a single biosample can be analyzed as analytical-target molecules.

As described, by the present invention, it is possible to establish a high-sensitive and high-speed mass spectrometric method by various types of stable isotopic element labels, and therefore, the present invention is applicable for all research fields where labeling is performed by fluorochrome or radioactive isotopic elements.

Further, according to the present invention, since the stable isotopic elements can be used without using radioactive isotopic element, facility to be used is not limited, installation in medical facility and private firms becomes possible.

### Effect of the Invention

The present invention exerts an excellent effect that time required for analysis can be shortened significantly comparing to conventional art.

Further, the present invention exerts an excellent effect that highly reliable result can be obtained avoiding sway of analytical result by observers.

Further, the present invention exerts an excellent effect that one-time analysis of a multiplicity of genes or the like can be performed on a single biosample, workload and time efficiencies can be improved, analysis of a plurality of genes or the like can be performed under conditions completely free from any difference in background attributed from biosample.

Further, the present invention exerts an excellent effect that it can eliminate the fear the analysis of mass spectrum becomes difficult when performing the mass spectrometry of molecules in the biosample, and high resolving power is not necessary in a mass spectrometer.

Further, the present invention exerts an excellent effect that the atomization and the ionization of constituting atoms constituting the molecules in the biosample can be simultaneously realized by one laser source, and a system constitution and laser irradiation control can be significantly simplified.

Further, the present invention exerts an excellent effect that efficient analysis can be performed even in the state where many types of labeled isotopes are mixed.

### BRIEF DESCRIPTION OF THE DRAWINGS

FIG. 1 is a conceptual constitution explanatory view of an analyzing apparatus example of a biotissue section by using laser ablation by the present invention, and is a conceptual constitution explanatory view of an analyzing apparatus of a biotissue section, which is constituted as a mass spectrometric system that performs the mass spectrometry of the molecules in a biotissue section by using laser ablation by the present invention.

FIG. 2 is an explanatory view of the principle of in situ hybridization by staining of marker genes by a TAS sensitization method.

FIG. 3 is an explanatory view of the principle of hybridization using simultaneous multiple probes.

FIG. 4 is an explanatory view of the principle of in situ hybridization by a single probe sensitization method.

FIG. 5 is an explanatory view showing the state where the biotissue section used in experiment is observed by a microscope.

FIG. 6 is a graph showing mass spectrum when ultra-short pulse laser beams are irradiated on region 1.

FIG. 7 is a graph showing mass spectrum when ultra-short pulse laser beams are irradiated on region 2.

FIG. 8 is a graph showing mass spectrum when ultra-short pulse laser beams are irradiated on region 3.

FIG. 9 is a graph showing mass spectrum when ultra-short pulse laser beams are irradiated on region 4.

FIG. 10 is a table showing labeled atomic weight, which is calculated based on peaks derived from each labeled atom from the mass spectrum shown in FIGS. 6 to 9.

FIG. 11 is an explanatory view in which regions having high gene strength in region 1 shown on FIG. 10 are shown surrounding by a dashed line on the explanatory view showing the state of region 1 observed by the microscope.

FIG. 12 is an explanatory view in which regions having high gene strength in region 2 shown on FIG. 10 are shown surrounding by a dashed line on the explanatory view showing the state of region 2 observed by the microscope.

FIG. 13 is an explanatory view in which regions having high gene strength in region 3 shown on FIG. 10 are shown surrounding by a dashed line on the explanatory view showing the state of region 3 observed by the microscope.

FIG. 14 is an explanatory view in which regions having high gene strength in region 4 shown on FIG. 10 are shown surrounding by a dashed line on the explanatory view showing the state of region 4 observed by the microscope.

FIG. 15 is an explanatory view showing a tissue where an expressed gene is detected by using an antisense probe and a sense probe of negative control regarding example 2, and an explanatory view showing graphs showing spectrum obtained by irradiating a laser and results where color is developed on the expression of the same gene on an adjacent section by the fixed method of nitro blue tetrazolium (NBT) for comparison.

### EXPLANATION OF REFERENCE NUMERALS

### 10 Analyzing apparatus

### 12 Vacuum tank

### 14 Target

### 16 Time-of-flight mass spectrometer (TOF mass spectrometer)

### 18 Rotational inlet terminal

### 20 Ultra-short pulse laser generating unit

### 22 Microscope unit

### 24 Image analysis apparatus

### BEST MODE FOR IMPLEMENTING THE INVENTION

### In the following, description will be made for an embodiment example of the method of analyzing a biosample using laser ablation and an apparatus therefor by the present invention in detail with reference to the attached drawings.

According to the method of analyzing a biosample using laser ablation and an apparatus therefor by the present invention, they are capable of analyzing the molecules in a biosample, and in the following explanation, a case where a biotissue section is used as the biosample to be analyzed will be described.

It is to be noted that, as a biosample to be analyzed in the method of analyzing a biosample using laser ablation and an apparatus therefor by the present invention, there are a smear sample of blood, saliva, sputum, urine or the like, a cultured cell, or an infection inspection, for example, other than the biotissue section.

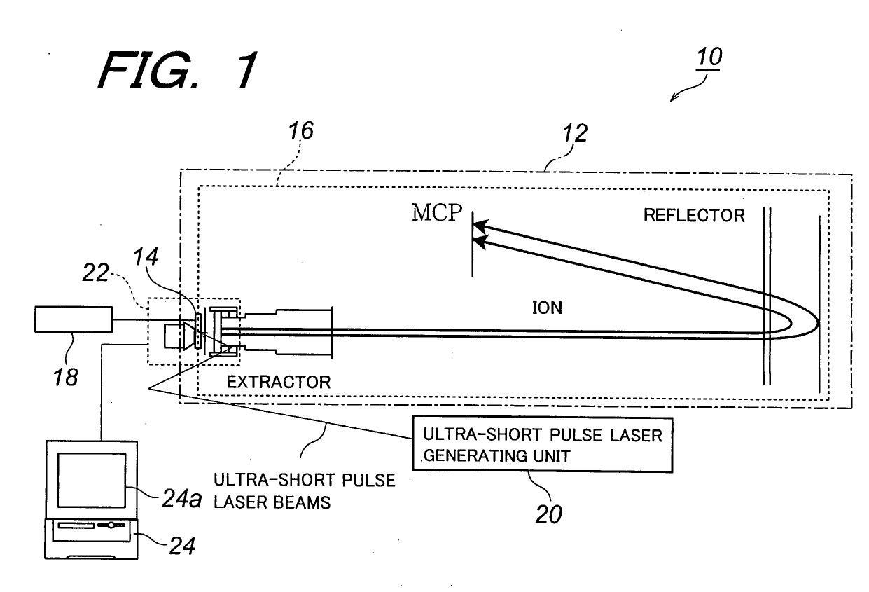

FIG. 1 shows the conceptual constitution explanatory view of the analyzing apparatus of a biotissue section, which is constituted as a mass spectrometric system that performs the mass spectrometry of the molecules in a biotissue section by using laser ablation by the present invention (hereinafter, simply referred to as an “analyzing apparatus” appropriately) as an embodiment example of the analyzing apparatus of biosample by using laser ablation by the present invention.

The analyzing apparatus 10 has: a vacuum tank 12 that can be set to the vacuum level of 10⁻⁸ to 10⁻⁶ Torr; a target 14 of a sample being the biotissue section to be analyzed that is arranged in the vacuum tank 12; a time-of-flight mass spectrometer (TOF mass spectrometer) 16 as a mass spectrometer arranged in the vacuum tank 12; a rotational inlet terminal 18 that rotates the target 14; an ultra-short pulse laser generating unit 20 that outputs ultra-short pulse laser beams such as a femto-second laser beam, for example, and irradiates it on the target 14; a microscope unit 22 for observing the target 14; and an image analysis apparatus 24 including a display section 24a, which analyzes the image of the target 14 observed by the microscope unit 22 and displays its analytical result.

It is to be noted that the ultra-short pulse laser beams irradiated from the ultra-short pulse laser generating unit 20 are focused on the target 14 via an optical system such as a focusing lens (not shown) and a mirror (not shown).

Herein, although not shown in detail, the microscope unit 22 may be either an upright-type microscope (upright microscope) unit or an inverted-type microscope (inverted microscope) unit.

Further, both of the observation by the microscope unit 22 and irradiation of the ultra-short pulse laser beams by the ultra-short pulse laser generating unit 20 maybe performed from the same surface of the target 14 or may be performed from different surfaces. In other words, the ultra-short pulse laser beams are irradiated on one surface of the target 14 from the ultra-short pulse laser generating unit 20, and the state of the target 14 may be observed from the one surface by the microscope unit 22. Alternatively, the ultra-short pulse laser beams are irradiated on one surface of the target 14 from the ultra-short pulse laser generating unit 20, and the state of the target 14 may be observed from the other surface different from the one surface by the microscope unit 22, so that the changing state of target 14 caused by the irradiation of the ultra-short pulse laser beams by the ultra-short pulse laser generating unit 20 can be observed in situ in real time.

Specifically, when the upright microscope is used as the microscope unit 22, the objective lens of the upright microscope unit is arranged on the upper surface of the target 14, and the irradiation of the ultra-short pulse laser beams from the ultra-short pulse laser generating unit 20 can be performed from the lower surface of the target 14.

Similarly, when the upright microscope is used as the microscope unit 22, the objective lens of the upright microscope is arranged on the upper surface of the target 14, and the irradiation of the ultra-short pulse laser beams from the ultra-short pulse laser generating unit 20 may be performed from the upper surface of the target 14. Furthermore, in this case, the irradiation of the ultra-short pulse laser beams may be performed through the objective lens of the upright microscope.

On the other hand, when inverted microscope is used as the microscope unit 22, the objective lens of the inverted microscope is arranged on the lower surface of the target 14, and the irradiation of the ultra-short pulse laser beams from the ultra-short pulse laser generating unit 20 can be performed from the upper surface of the target 14.

Similarly, when the inverted microscope is used as the microscope unit 22 the objective lens of the inverted microscope is arranged on the lower surface of the target 14, and the irradiation of the ultra-short pulse laser beams from the ultra-short pulse laser generating unit 20 may be performed from the lower surface of the target 14. Furthermore, in this case, the irradiation of the ultra-short pulse laser beams may be performed through the objective lens of the inverted microscope.

In other words, when the ultra-short pulse laser beams are irradiated from the ultra-short pulse laser generating unit 20 on one surface of the target 14 and the state of the target 14 is observed from the one surface of the target 14 by the microscope unit 22, arrangement may be done in such a manner that the irradiation of the ultra-short pulse laser beams are performed through the objective lens of the microscope unit 22.

Further, as the ultra-short pulse laser generating unit 20, a unit such as a femto-second laser, for example, capable of irradiating the ultra-short pulse laser beams whose pulse time width is 1 femto second or more and 1 pico second or less, and whose peak value output is 1 mega watt or more and 10 giga watts or less can be used.

More particularly, such an ultra-short pulse laser generating unit 20 is constituted of a titanium sapphire laser, for example, and a unit having parameters shown below can be used. Specifically, they are as follows.

Peak width (pulse time width): up to 110 fs (femto seconds)

Output: 50 to 480 μJ (microjoule)

(peak value output: 0.5 to 4 GW (giga watts))

Wavelength: up to 800 nm (nanometers)

Repetition: 1 kHz (kilohertz)

Further, it goes without saying that various types of mass spectrometers such as a quadrupole mass spectrometer may be used as the mass spectrometer instead of the time-of-flight mass spectrometer.

Further, the focal distance of the focusing lens that focuses the ultra-short pulse laser beams outputted from the ultra-short pulse laser generating unit 20 is set to 25 cm, for example.

In the above-described constitution, description will be made for a method of performing mass spectrometry of molecules in the biotissue section by using the above-described analyzing apparatus 10.

Herein, in the mass spectrometry of molecules in the biotissue section by the present invention, mass spectrometry of molecules in the biotissue section is performed by using the ablation by the ultra-short pulse laser beams outputted from the ultra-short pulse laser generating unit 20 such as the femto-second laser and the analysis by the time-of-flight mass spectrometer 16.

In other words, the mass spectrometry of molecules in the biotissue section by the present invention, by irradiating the ultra-short pulse laser beams on the biotissue section to be analyzed and ablating the biotissue section, atomizes the molecules contained in the biotissue section into constituting elements, ionizes the atomized constituting elements, and analyzes the ionized constituting elements.

Specifically, the biotissue section is arranged in the vacuum tank 12 as the target 14, the ultra-short pulse laser beams such as a femto-second laser beam outputted from the ultra-short pulse laser generating unit 20 is irradiated on the biotissue section being the target 14 to perform ablation, and analysis is done by the time-of-flight mass spectrometer 16.

In this occasion, when labels are applied to the molecules in the biotissue section to be analyzed by element labels or the like, the biotissue section containing labeled molecules is arranged in the vacuum tank 12 as the target 14, the ultra-short pulse laser beams such as the femto second laser beam outputted from ultra-short pulse laser generating unit 20 are irradiated on the biotissue section being the target 14 to perform ablation, the labeled elements are measured by the time-of-flight mass spectrometer 16, and thus the molecules in the biotissue section to be analyzed can be detected and analyzed.

Herein, the irradiation position of the ultra-short pulse laser beams such as the femto second laser beam outputted from ultra-short pulse laser generating unit 20 can be determined in advance by observing the target 14 being the biotissue section by the microscope unit 22.

Moreover, an image at each point, where laser irradiation was performed on the target 14 being the biotissue section obtained by the microscope unit 22, is analyzed by the image analysis apparatus 24, the strength of labeled element at each point is displayed on the biotissue section image being the target 14 in a converted state into chromatic display on the display section 24a of the image analysis apparatus 24, a processing to allow the morphologic characteristics of the biotissue section and the measurement result to be simultaneously recognized is conducted, and thus data can be analyzed in the same style as a conventional in situ hybridization method.

Further, before setting the biotissue section as the target 14 on the analyzing apparatus 10 that performs ablation by the ultra-short pulse laser beams, the biotissue section is observed by the microscope unit 22 in advance, a reference point is provided on the biotissue section, a laser irradiation position is positioned by using the point as a reference, and thus an observed image by the microscope unit 22 and an analytical result of molecules measured by the ablation using the ultra-short pulse laser beams can be corresponded with each other.

It is to be noted that, in the present invention, molecules themselves that are labeled by isotopic elements are ionized on an atomic level and labeled elements can be detected, so that an applicable range of mass spectrometry can be dramatically widened. For example, isotopic elements can be used as labels, and the types of label can be increased to as many as 270 that is the number of stable isotopic elements, for example. Thus, amount of information can be dramatically increased comparing to the fluorescence method (2 types) and the radioactive isotopic elements (about 10 types) being conventional labeling methods.

### EXAMPLE 1

In the following, description will be made for a method of fabricating a biotissue section, which is formed by slicing a mouse brain, as an example of fabricating a biosample.

### 1. Preparation of Mouse Brain

Animal: As a model animal, 10 to 11 week-old male wild-type CD-1 mice and C57BL/6J purchased from Oriental Yeast Co., Ltd. were used.

Fixative as a reagent when preparing a mouse brain is as shown in the following table 1.

TABLE 1
Reagent: fixative
Neutral fixative: 1 L 4% PFA (para form aldehyde) liquid (pH 7.5)

| | | |
|---|---|---|
| PFA | 40 | g |
| Sucrose | 40 | g |
| Na₂HPO₄•12H₂O | 11.4 | g |
| NaH₂PO₄•2H₂O | 3.3 | g |

Acidic fixative: Original-Bouin liquid (pH 3.5 to 4.0)

| | | |
|---|---|---|
| Saturated picric acid | 300 | ml |
| Formalin | 100 | ml |
| Acetic acid | 20 | ml |

By using the above-described reagent, a paraffin section and a frozen section of the mouse brain were fabricated as biotissue sections by the following method.

<Method>

1) Fabricating a paraffin section

After anesthetizing a mouse by ether to eliminate pain response, an abdominal cavity was opened to expose a heart, 20 ml each of ice-cooled neutral fixative and acidic fixative were sequentially poured into the heart to perform perfusion fixation.

After that, the brain was removed, it was left to stand for 3 days in acidic fixative at 4° C. and fixed.

The fixed tissue was sliced to fabricate a section, and it was adhered on a glass slide. A paraffin section was fabricated by using an automatic in situ hybridization (in situ hybridization) unit manufactured by Ventana Medical Systems Inc.

2) Fabricating a frozen section

The brain was removed after dislocating a spinal cord under ether anesthesia, embedded in OCT and frozen by liquid nitrogen, and a section was created by a slicer manufactured by Leica Microsystems.

### 2. Designing of Primer

The cDNA sequence of a target gene was searched from RIKEN FANTOM clone. Furthermore, cDNA was referred to by the public database of LoucusLink and a target cDNA sequence was selected. The selected base sequence was transformed into an amino-acid sequence, and homology search was conducted by an NCBI Protein BLAST. A cDNA area corresponding to a low homology sequence was identified, and a primer was designed in order to amplify it as a template. Furthermore, depending on needs, a primer was designed in order to amplify a low homology sequence from a genome DNA as a template. Now, Table 2 shows the LoucusLink IDs of genes.

TABLE 2

| Gene name | Locus Link ID |
|---|---|
| Tph2 | NM_173391 |
| MaoB | NM_172778 |
| AADC | NM_016672 |
| Htr1B | NM_010482 |

### 3. Plasmid Purification

Spectrophotometer (Bio Spec-1600) manufactured by Shimadzu Corporation and GeneAmp PCR System 9700 manufactured by Applied Biosystems were used as equipment, and reagent shown in Table 3 was used, and plasmid purification was performed by the following method.

TABLE 3
Reagent

QIAprep Spin Miniprep Kit: QIAGEN
TaKaRa EX Taq (trademark): TaKaRa
MicroSpin (trademark) Column: Amersham Pharmacia Biotech
MinElute Gel Extraction Kit: QIAGEN
DIG RNA Labeling Kit (SP6/T7): Roche Diagnostics
SP6/T7 Transcription Kit: Roche Diagnostics
BD CHROMA SPIN Column: Clontech

### Method

A plasmid DNA was purified from coliform bacillus by using the QIAprep Spin Miniprep Kit according to the attached protocol, absorbance was measured by the spectrophotometer, and concentration was calculated. PCR was performed by using a primer that was designed by using the Plasmid DNA as a template. Composition of reaction liquid and reaction conditions are shown below. Electrophoresis was applied to a reaction product in 2% agarose gel and confirmed.

### 4. Probe Fabrication

1) Fabricating a stable isotopic labeling ssDNA probe

The stable isotopic labeling ssDNA probe was fabricated by the reaction liquid and the reaction condition shown in Table 4. Eu-labeled dUTP, Sm-labeled dUTP, Tb-labeled dUTP and Dy-labeled dUPT manufactured by PerkinElmer, Inc. were used as a labeled dNTP.

TABLE 4
Composition of reaction liquid

| | μl |
|---|---|
| Plasmid DNA (1 ng/μl) | 0.5 |
| F primer (25 μM) | 1.0 |
| 10xBuffer | 5.0 |
| Labeled dNTP | 1.0 |
| dNTP Mixture | 4.0 |
| TaKaRa Ex Taq (trademark) (5 units/μl) | 0.5 |
| Sterilized water | 34.0 |
| | 50.0 |

Reaction condition

| | | |
|---|---|---|
| 95° C. | 5 minutes | 30 cycles |
| 95° C. | 1 minute | |
| 60° C. | 1 minute | |
| 72° C. | 1 minute | |
| 72° C. | 3 minutes | |
| 4° C. | ∞ | |

Phenol-chloroform extraction was performed to a PCR product and it was purified by the MicroSpin (trademark) column. The purified product was confirmed on agarose gel.

2) Fabricating a DIG-labeled RNA probe

A DIG-labeled RNA probe was fabricated by the reaction liquid and the reaction condition shown in Table 5.

TABLE 5
Composition of reaction liquid

| | μl |
|---|---|
| Plasmid DNA (1 ng/μl) | 0.5 |
| F primer (25 μM) | 1.0 |
| R primer (25 μM) | 1.0 |
| 10xBuffer | 5.0 |
| dNTP Mixture | 4.0 |
| TaKaRa EX Taq (trademark) (5 units/μl) | 0.5 |
| Sterilized water | 34.0 |
| | 50.0 |

Reaction condition

| | | |
|---|---|---|
| 95° C. | 5 minutes | 30 cycles |
| 95° C. | 1 minute | |
| 60° C. | 1 minute | |
| 72° C. | 1 minute | |
| 72° C. | 3 minutes | |
| 4° C. | ∞ | |

By using the PCR product and a primer containing the recognition sequence of T7 and SP6 polymerase (*T7, SP6 adaptor), PCR was performed again. The reaction liquid composition and the reaction condition are shown on Table 6. Electrophoresis was applied to a reaction product in 2% agarose gel and confirmed.

| | |
|---|---|
| *T7 adaptor: | GAGCGCGCGTAATACGACTCACTATAGGGC |
| SP6 adaptor: | TTGTGCGGCCATTTAGGTGACACTATAGAA |

TABLE 6
Composition of reaction liquid

| | μl |
|---|---|
| 1st PCR product | 0.5 |
| SP6 adaptor (10 μM) | 2.5 |
| T7 adaptor (10 μM) | 2.5 |
| 10xBuffer | 5.0 |
| dNTP Mixture | 4.0 |
| TaKaRa Ex Taq (trademark) (5 units/μl) | 0.5 |
| Sterilized water | 34.0 |
| | 50.0 |

Reaction condition

| | | |
|---|---|---|
| 95° C. | 5 minutes | 25 cycles |
| 95° C. | 1 minute | |
| 60° C. | 1 minute | |
| 72° C. | 1 minute | |
| 72° C. | 3 minutes | |
| 4° C. | ∞ | |

Phenol-chloroform extraction was performed to a PCR product and it was purified by the MicroSpin (trademark) column.

Next, description will be made for the fabrication of a DIG-labeled RNA antisense probe and sense probe.

In other words, T7 polymerase was used in fabricating the antisense probe, SP6 polymerase was used in fabricating the sense probe, and they were incubated in the composition of reaction liquid shown in Table 7 at 37° C. for 2 hours. Electrophoresis was applied to a reaction product in 1% agarose gel and confirmed.

TABLE 7
Composition of reaction liquid

| | μl |
|---|---|
| PCR product | 10.0 |
| 10xTranscription buffer | 2.0 |
| NTP labeling mixture | 2.0 |
| RNase inhibitor (40 U/μl) | 0.5 |
| T7 or SP6 polymerase (20 U/μl) | 2.0 |
| Sterilized water | 3.5 |
| | 20.0 |

After the confirmation, 2 μl of DNaseI (10 U/μl) was added to remove DNA, and they were incubated at 37° C. for 30 minutes. Whether or not the DNA was removed was confirmed by performing electrophoresis in 1% agarose gel. When it was removed, 2 μl of 0.2 M EDTA was added to stop reaction, and the probes were purified in a BD CHROMA SPIN Column.

### 5. Determination of Probe By Dot-plot Method

Accurately determining the obtained labeled DNA after the fabrication of probe is extremely important for optimizing the result of ISH and giving reproducibility. Now, diluted standard having a known amount, dilution series of an unknown amount of actually labeled probe, and dilution array of specimen are parallelly spotted, and they were examined by a laser irradiation method and a color development method using an anti-DIG antibody.

In the following, the determination of DIG-labeled probe will be described. First, equipment and reagent to be used will be described.

A shaker, a UV crosslinker and a nylon membrane (Amersham Pharmacia Biotech Hybond-N) were used as equipment, reagent as shown in Table 8 was used, and the determination of the DIG-labeled probe was performed by the following method.

TABLE 8
Reagent

Control DIG-label DNA: Roche Diagnostics
TBS buffer
0.5M Tris-Cl (pH 7.4)
1.5M NaCl
Blocking reagent
TBS-T (TBS, 0.1% Tween20)
5% skim milk
APB buffer (4° C.)
100 mM Tris-Cl (pH 9.5)
50 mM MgCl₂
100 mM NaCl
Anti-DIG-AP: Roche Diagnostics, Lot1 093 274
NBT/BCIP solution
(Nitro blue tetrazolium/bromo-chloro-indryl-phosphate)

<Method>

Dilution series of control RNA and of the fabricated probe was made on a 96-well plate, and diluted solution of each well was spotted on nylon membrane by 1 μl. After air-drying the membrane, it was processed (120 mJ/cm²) by a UV crosslinker. After the membrane was soaked in solution added with the blocking reagent and shaken by a shaker for 10 minutes, it was shaken by 5000-fold diluted liquid of Anti-DIG-AP for 30 minutes to perform the antibody reaction. After cleaning the membrane by TBS buffer, NBT/BCIP solution was added and shaken to perform color-developing reaction. After color was fully developed, MilliQ water was added and shaken for 10 minutes to stop the color-developing reaction. Then, it was washed by running water and air-dried, hermetically sealed by hybripack, spot signals of the control RNA and the fabricated probe were compared, and the determination of probe was performed.

### 6. In Situ Hybridization Method

1) Hybridization of marker gene

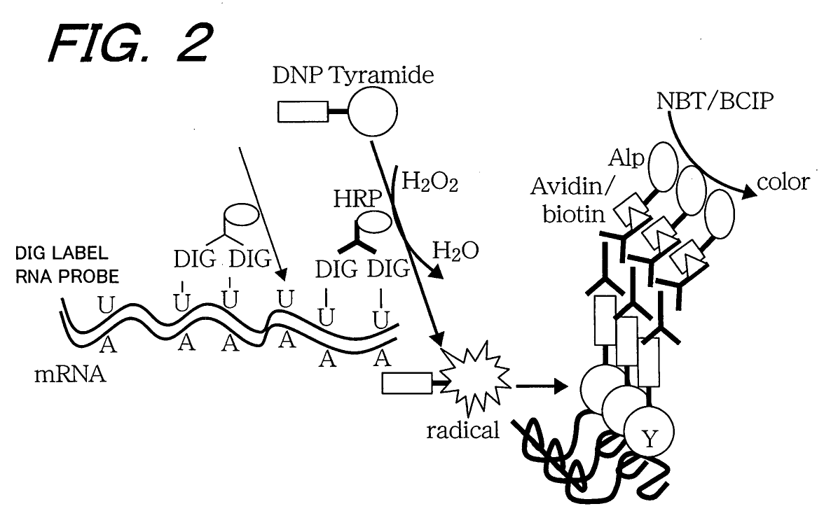

As shown in FIG. 2, the DIG-labeled RNA probe was used as a probe, and color development by alkaline phosphatase was performed by an automatic ISH unit manufactured by Ventana Medical Systems Inc.

2) Hybridization using simultaneous multiple probes

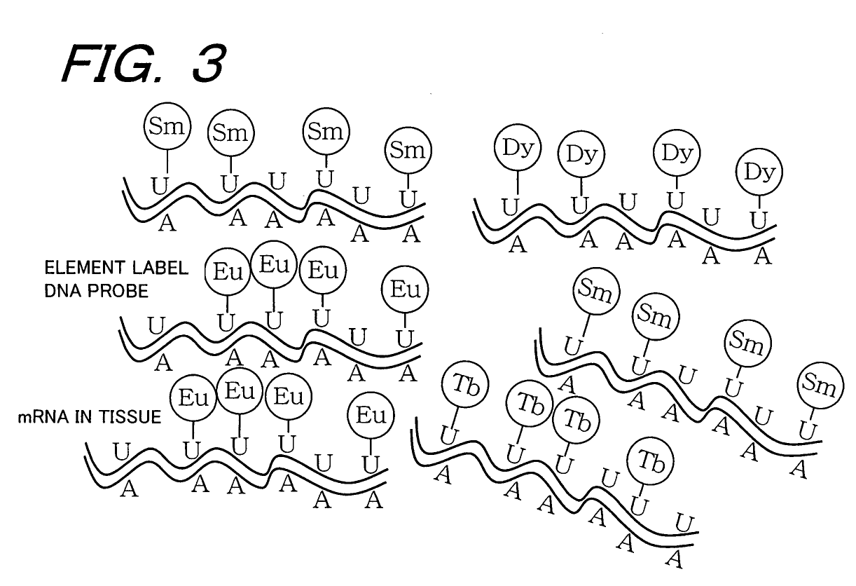

As shown in FIG. 3, after labeling isotopic elements (Eu, Sm, Tb, Dy) to the probes and performing pre-processing by the automatic ISH unit, hybridization was performed in short time by using a SAW agitation method (Advalytix).

Map2 marker gene DIG label

Tph2 target gene 1 Sm-labeled NM_{—}173391

MaoB target gene 2 Eu-labeled NM_{—}172778

AADC target gene 3 Dy-labeled NM_{—}016672

Htr1B target gene 4 Tb-labeled NM_{—}010482

3) High-sensitive hybridization

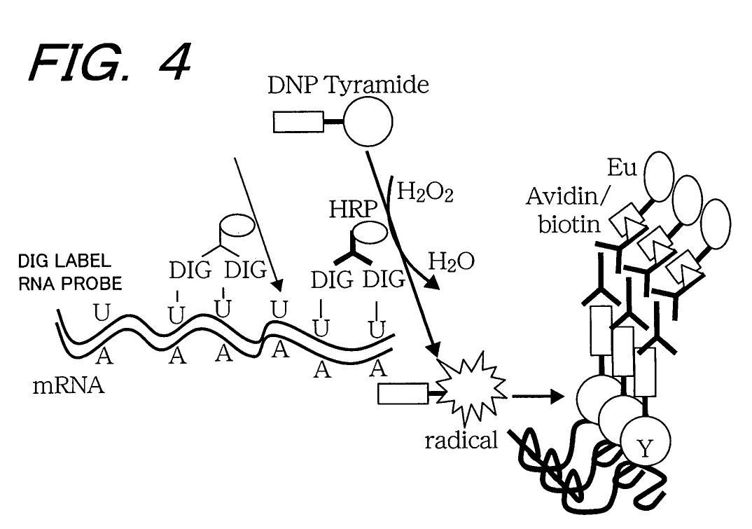

As shown in FIG. 4, by using the DIG-labeled RNA probe as a probe, high-sensitive hybridization was performed by using a sensitization method that combines a tyramid sensitization method and avidinated Eu.

In the following, description will be made for a result obtained by using a biotissue section as a biosample, irradiating the ultra-short pulse laser beams on the biotissue section to perform ablation, and analyzing the biotissue section.

In other words, the experiment result of an analysis performed by the analyzing apparatus 10, where the biotissue section of the mouse brain obtained by the method described in the above-described 1 to 4 was used as the target 14, is shown as follows.

This experiment is that the biotissue section fabricated as described above is used as the target 14, the ultra-short pulse laser beams such as the femto-second laser beam from the ultra-short pulse laser generating unit 20 are irradiated to perform ablation, and it is analyzed by the time-of-flight mass spectrometer 16.

More particularly, the target 14 fabricated as described above is installed in the vacuum tank 12, the inside of the vacuum tank 12 is evacuated such that the vacuum level in the vacuum tank 12 becomes 10⁻⁶ Torr or less.

Next, the ultra-short pulse laser beams outputted from the ultra-short pulse laser generating unit 20 are focused on the target 14 by using an optical system such as the focusing lens, and a region set on the target 14 is ablated.

Then, the mass of monovalent ions generated by the irradiation of the ultra-short pulse laser beams onto the target 14 is measured by the time-of-flight mass spectrometer 16.

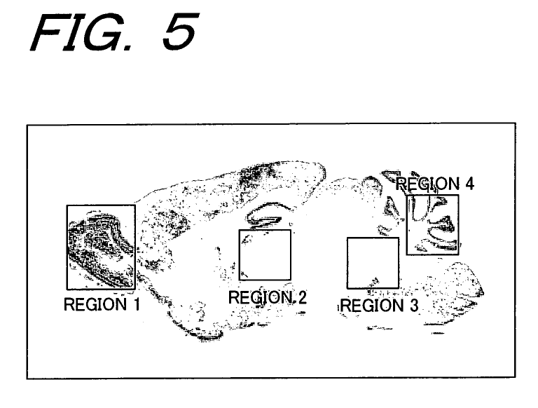

Herein, FIG. 5 is the explanatory view showing the state where the biotissue section used in the experiment was observed by the microscope unit 22, where the localization of a marker gene is shown by using a TSA sensitization ISH method. When this probe is used, a nerve cell can be selectively stained, and its signal becomes a marker when performing laser irradiation.

By using the position of the marker gene as an origin, regions (1 to 4) surrounded by squares in FIG. 5 are set, and the irradiation of the ultra-short pulse laser beams was performed by the ultra-short pulse laser generating unit 20.

Conditions in irradiating the ultra-short pulse laser beams are as follows.

Pulse width: 100 fs (femto seconds)

Laser power: 0.2 mJ (millijoule)

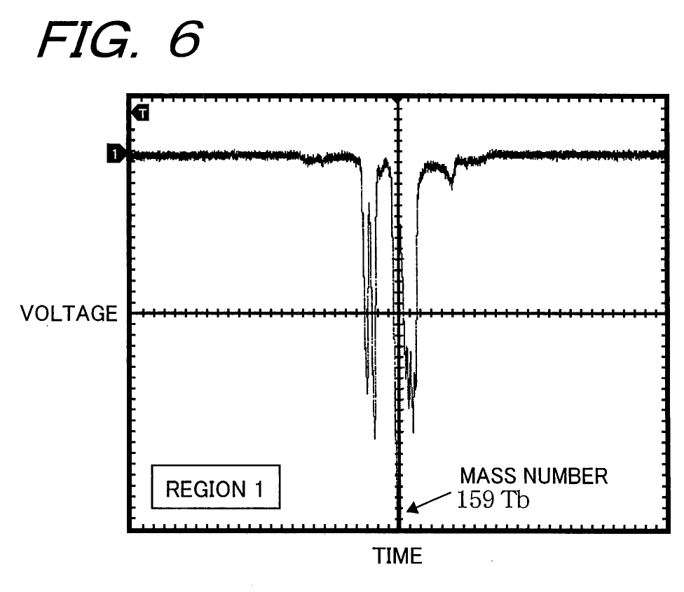

The ultra-short pulse laser beams were irradiated onto each of the regions (1 to 4) on the above-described irradiation conditions, ions generated by the irradiation of the ultra-short pulse laser beams were measured by the time-of-flight mass spectrometer 16, and labeled elements in each region were measured, and the mass spectrum shown in FIGS. 6 to 9 was obtained.

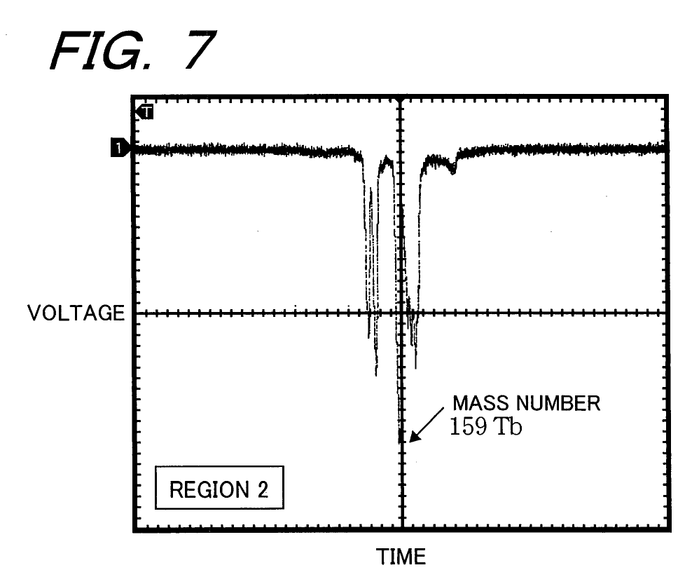

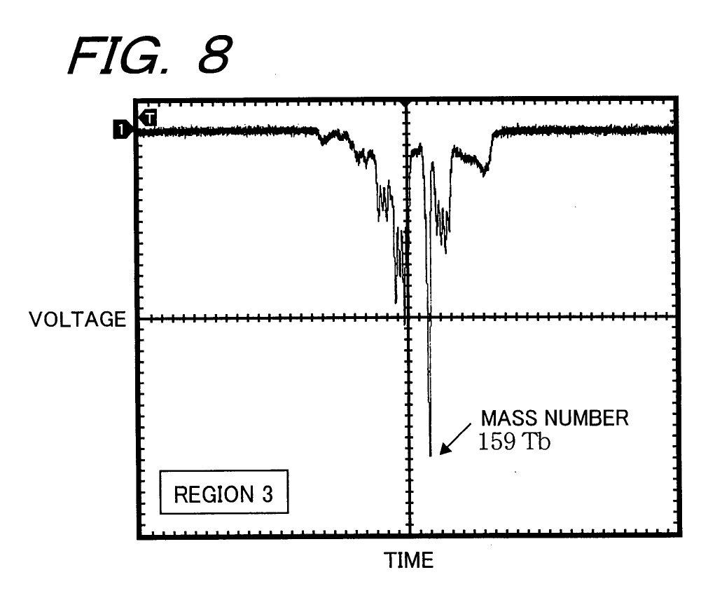

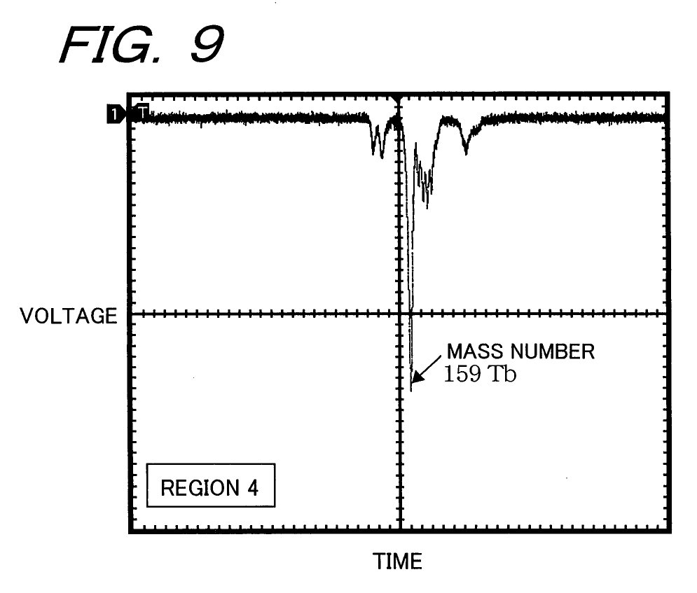

Specifically, FIG. 6 shows the mass spectrum when the ultra-short pulse laser beams were irradiated on the above-described region 1, FIG. 7 shows the mass spectrum when the ultra-short pulse laser beams were irradiated on the above-described region 2, FIG. 8 shows the mass spectrum when the ultra-short pulse laser beams were irradiated on the above-described region 3, and FIG. 9 shows the mass spectrum when the ultra-short pulse laser beams were irradiated on the above-described region 4.

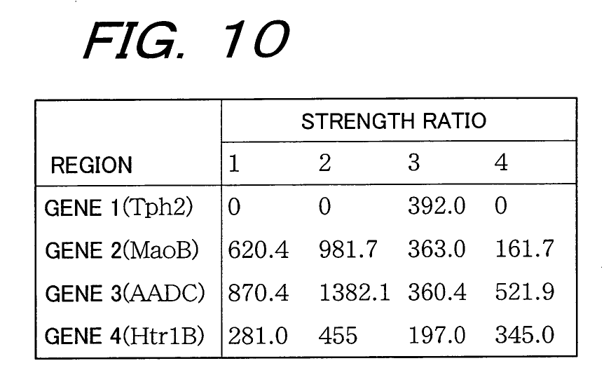

From the above-described mass spectrum shown in FIGS. 6 to 9, a table shown as FIG. 10 summarizes labeled atomic weight that was calculated based on peaks derived from each labeled atom.

As shown in FIG. 10, by irradiating the ultra-short pulse laser beams such as the femto-second laser beam on the biotissue section and measuring the labeled atom, the expression of gene in the biotissue section could be analyzed.

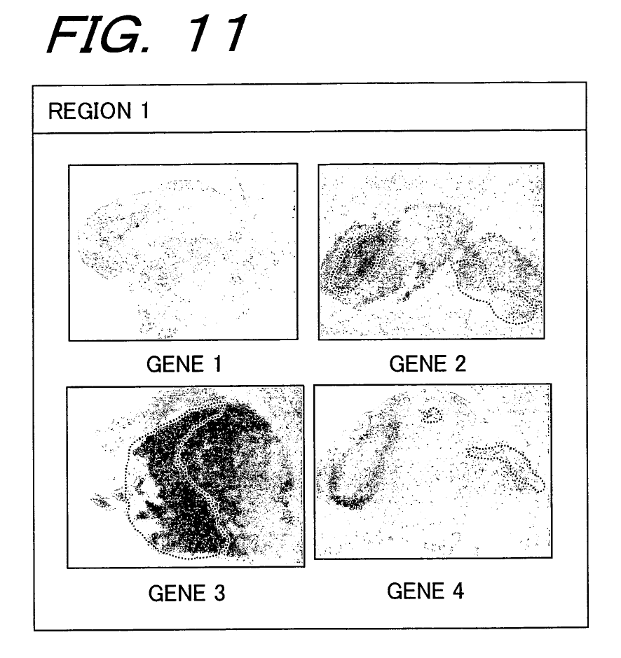

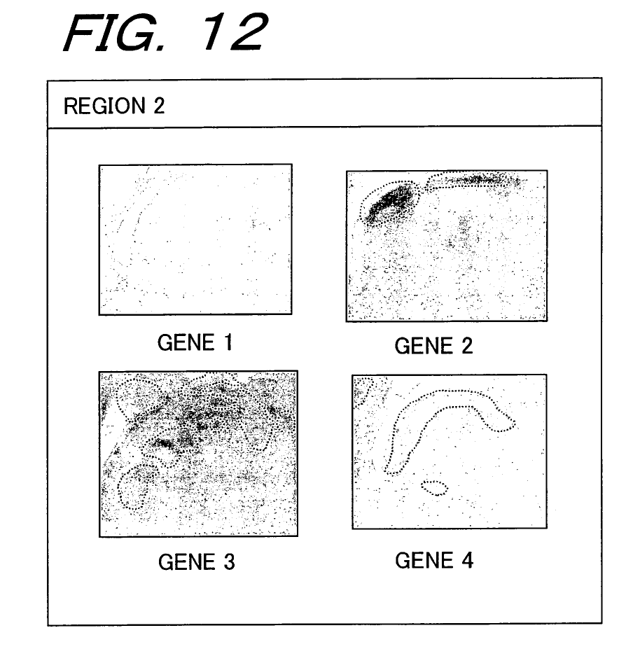

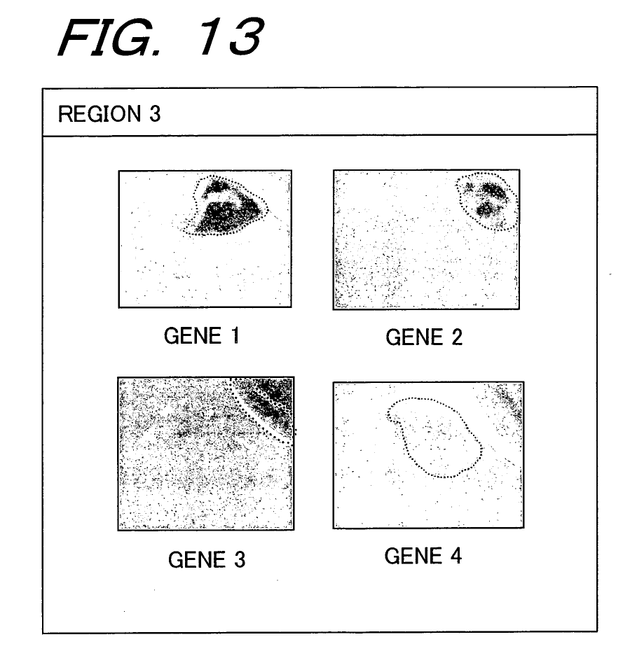

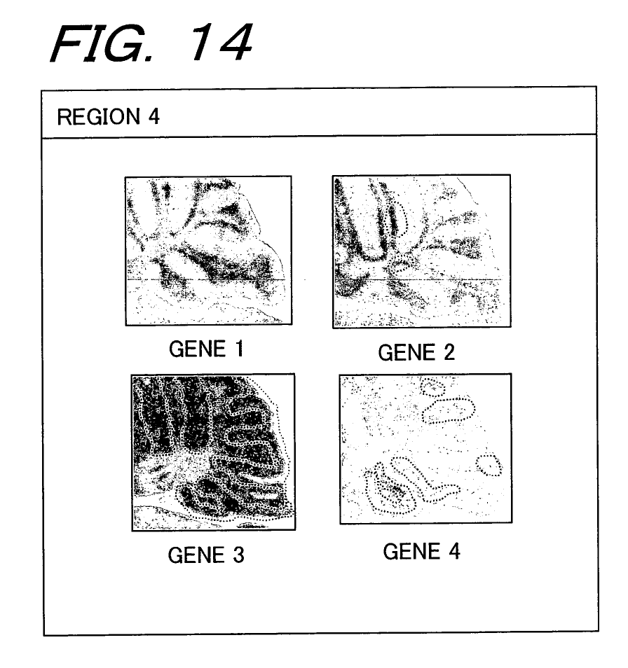

Further, FIGS. 11 to 14 are the explanatory views showing the state of the regions (1 to 4) shown on FIG. 10 observed by the microscope, on which regions having high gene strength in the regions (1 to 4) are shown by encircling with dashed lines, by using the microscope unit 22 and the image analysis apparatus 24. It is to be noted that the strength distribution can be shown in more detail when color images are used as images.

As shown in FIGS. 11 to 14, according to the analyzing apparatus 10, the images of the regions (1 to 4) obtained by the microscope unit 22, on the target 14 being the biotissue section to which the laser irradiation was performed, is analyzed by the image analysis apparatus 24, displayed on the display section 24a of image analysis apparatus 24, the strength of labeled elements in the regions (1 to 4) is displayed on the section images of the regions (1 to 4) in the form transformed into closing line display or chromatic display, and the morphologic characteristics of the biotissue section and the measurement result can be recognized at the same time.

It is to be noted that, in this example, an image for each gene was obtained for each region, that is, 4 images for genes (1 to 4) were obtained for each region of the regions (1 to 4), but it goes without saying that the invention is not limited to this. The expression of a plurality of genes may allowed to be shown on the image of each region by changing the color of gene using color images.

### EXAMPLE 2

Next, description will be made for detection using a Pt-labeled RNA probe.

The expression of the gene of microtubule-associated protein MAP2, which exists on dendrite in a large volume, in the mouse brain was investigated.

It is to be noted that the preparation of the mouse brain, the design of the primer, and the purification of plasmid were performed in the same manner as “Example 1”.

### 1. Fabricating a Probe

1) Fabricating Pt-labeled RNA probe

A template DNA was created by the reaction liquid and reaction condition shown in FIG. 5. Further, an RNA probe was fabricated.

By using the PCR product and the primer containing the recognition sequence (*T7, SP6 adaptor) of T7 and SP6 polymerase, PCR was performed again. The reaction liquid composition and the reaction condition are as shown in Table 6. Electrophoresis was applied to the reaction product in 2% agarose gel and confirmed.

| | |
|---|---|
| *T7 adaptor: | GAGCGCGCGTAATACGACTCACTATAGGGC |
| SP6 adaptor: | TTGTGCGGCCATTTAGGTGACACTATAGAA |

Phenol-chloroform extraction was conducted to the PCR product and it was purified by a MicroSpin (trademark) column.

Next, by using T7 polymerase in fabricating an antisense probe and by using SP6 polymerase in fabricating a sense probe, and they were incubated in the composition of reaction liquid shown in Table 9 at 37° C. for 2 hours. Electrophoresis was applied to a reaction product in 1% agarose gel and confirmed.

TABLE 9
Composition of reaction liquid

| | μl |
|---|---|
| PCR product | 10.0 |
| 10xTranscription buffer | 2.0 |
| NTP mixture | 2.0 |
| RNase inhibitor (40 U/μl) | 0.5 |
| T7 or SP6 polymerase (20 U/μl) | 2.0 |
| Sterilized water | 3.5 |
| | 20.0 |

After the confirmation, 2 μl of DNaseI (10 U/μl) was added to remove DNA, and they were incubated at 37° C. for 30 minutes. Whether or not the DNA was removed was confirmed by performing electrophoresis in 1% agarose gel. When it was removed, 2 μl of 0.2M EDTA was added to stop reaction, and the probes were purified in a BD CHROMA SPIN Column.

The purified RNA was labeled by using a ULYSIS Nucleic Acid Labeling Kit (manufactured by Molecular Probe Inc.). After 1/10 volume of 3M NaAcO (pH 5.2) and two-fold volume of ethanol were added to 1 μg of the purified RNA and left it to stand at −70° C. for 30 minutes, it was centrifuged on 12000 rpm at 4° C. for 15 minutes.

After washing a pellet by 70% ethanol and air-drying it, it was dissolved in 20 μl of labeling buffer (Component C). This was incubated at 95° C. for 5 minutes and placed on ice. By spinning it down to collect water droplets on the bottom of a tube, 5 μl of ULS labeling reagent (Alexa Fluor 532) was added, the labeling buffer (Component C) was added to make the total volume of 25 μl. After incubating at 90° C. for 10 minutes, it was placed on ice to stop reaction, and water droplets were collected on the bottom of the tube by spinning it down. 100 μl of TE was added to the labeled sample, and it was purified by a MicroSpin S-400 to form a Pt-labeled RNA probe.

### 2. In Situ Hybridization

Similar to Example 1, hybridization reaction was performed by using the automatic in situ hybridization (in situ hybridization) unit manufactured by Ventana Medical Systems Inc.

20 μl of the RNA probe was used for the in situ hybridization.

### 3. Detecting Pt Label By Using Time-of-flight Mass Spectrometer

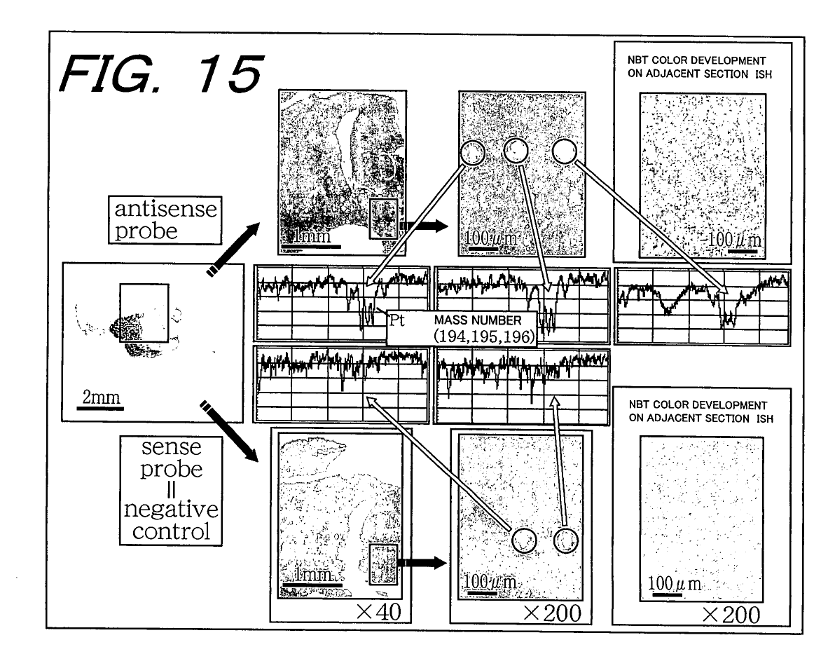

FIG. 15 shows the result of analysis performed by the analyzing apparatus 10 in the same method as Example 1.

FIG. 15 shows the explanatory view showing a tissue where the expressed gene is detected by using the antisense probe and the sense probe of negative control which were fabricated above, the spectrum obtained by irradiating a laser, and the result where color is developed on the expression of the same gene on an adjacent section by the fixed method of nitro blue tetrazolium (NBT) for comparison.

In the case of using the Pt-labeled antisense probe, peaks (mass number 194, 195, 196) derived from Pt are observed, while no peak of Pt was detected in the negative control.

This matches well the result by a conventional staining method, which is shown for comparison, and it was made clear that the analysis using the Pt-labeled antisense probe was useful in analyzing the expression of gene.

### Modified Example

It is to be noted that the above-described embodiments can be modified as shown in (1) to (12) below.

(1) In the above-described embodiments, the time-of-flight mass spectrometer that performs mass spectrometry by measuring the time of flight of atoms was used as a mass spectrometer, and mass spectrometry of a plurality of atoms can be performed simultaneously by one-time laser irradiation when the time-of-flight mass spectrometer is used. Further, even in the case where the ion cyclotron Fourier transform mass spectrometer is used as the mass spectrometer, mass spectrometry of a plurality of atoms can be performed simultaneously.
(2) In the above-described embodiments, description was made for mass spectrometry as an analysis method of molecule, but it goes without saying that the invention is not limited to this and the present invention may be used for analysis other than mass spectrometry.
(3) In the above-described embodiments, the rotational inlet terminal 18 that rotates the target 14 was used as the moving means for moving the target 14, but it goes without saying that the invention is not limited to this and appropriate moving means such as a freely movable table capable of mounting the target 14 thereon may be used.
(4) In the above-described embodiments, the target 14 was ablated without omission and duplication by rotating the target 14 with the use of the rotational inlet terminal 18, but it goes without saying that the invention is not limited to this and moving means for moving an irradiation position of the ultra-short pulse laser beams on the target may be provided to ablate the target 14 without omission and duplication.
(5) In the above-described embodiments, hybridization was shown as an example where the nucleic acid probe bonded to a particular target, but it goes without saying that the invention is not limited to this and bond such as aptamer is also acceptable other than hybridization.

### (6) In the present invention, labeling of nucleic acid for detecting target nucleic acid to be analyzed may be labeled by the TUNEL method.

### (7) Nucleic acid used as a probe in the present invention contains DNA, RNA, PNA, and other modified ones.

### (8) In the present invention, particular protein contained in a biotissue section may be detected.

### (9) In the present invention, labeled substances having specific bond to detect target protein may be allowed to bond by antigen-antibody reaction.

### (10) In the present invention, in the case where the nucleic acid is used as a label, the target naturally contains nucleic acid, and also contains protein or other low-molecular substances other than nucleic acid.

### (11) As a detection target in the present invention, DNA, RNA, protein, low-molecular compound, and other substances contained in samples are considered.

### (12) The above-described embodiments and the above-described modifications shown in (1) to (11) may be appropriately combined.

### INDUSTRIAL APPLICABILITY

The present invention is utilized in the analysis of biotissue in the field of life science such as medical science and biochemistry.

1. A method of analyzing a biosample using laser ablation, wherein
by irradiating ultra-short pulse laser beams on a biosample to be analyzed and ablating the sample, molecules contained in said biosample are atomized into constituting element, said atomized constituting elements are ionized, said ionized constituting elements are analyzed, and molecules to be analyzed in said biosample are analyzed.

2. The method of analyzing a biosample using laser ablation according to claim 1, wherein
by directly or indirectly labeling a substance having specific bond to molecules to be analyzed in said biosample and analyzing the molecules to which said labeled substance is bonded, molecules to be analyzed in said biosample are analyzed.

3. The method of analyzing a biosample using laser ablation according to claim 2, wherein
said labeled substance having specific bond is nucleic acid.

4. The method of analyzing a biosample using laser ablation according to any one of claims 1, 2 and 3, wherein
the molecules to be analyzed in said biosample are nucleic acid.

5. The method of analyzing a biosample using laser ablation according to claim 3, wherein
the nucleic acid being said labeled substance having specific bond contains DNA, RNA, PNA, and other modified acid.

6. The method of analyzing a biosample using laser ablation according to any one of claims 2, 3, 4 and 5, wherein
said labeled substance having specific bond is bonded by hybridization.

7. The method of analyzing a biosample using laser ablation according to any one of claims 2, 3, 4 and 5, wherein
said labeled substance having specific bond is aptamer.

8. The method of analyzing a biosample using laser ablation according to any one of claims 3, 4, 5, 6 and 7, wherein
the labeling of said nucleic acid is performed by a TUNEL method.

9. The method of analyzing a biosample using laser ablation according to any one of claims 1 and 2, wherein
the molecules to be analyzed in said biosample are protein.

10. The method of analyzing a biosample using laser ablation according to claim 9, wherein
said labeled substance having specific bond, which is used for analyzing said protein, is bonded by antigen-antibody reaction.

11. The method of analyzing a biosample using laser ablation according to any one of claims 2, 3, 4, 5, 6, 7, 8, 9 and 10, wherein
said label is an element label.

12. The method of analyzing a biosample using laser ablation according to claim 11, wherein
said element label is a stable isotopic element label.

13. The method of analyzing a biosample using laser ablation according to any one of claims 1, 2, 3, 4, 5, 6, 7, 8, 9, 10, 11 and 12, wherein
the analysis of said ionized constituting element is mass spectrometry.

14. The method of analyzing a biosample using laser ablation according to claim 13, wherein
said mass spectrometry is mass spectrometry by a time-of-flight method.

15. The method of analyzing a biosample using laser ablation according to any one of claims 2, 3, 4, 5, 6, 7, 8, 9, 10, 11, 12, 13 and 14, wherein
multi-channeling is conducted by using plural types of labels as labels, and at least 2 types or more molecules in a single biosample are analyzed as analytical-target molecules.

16. The method of analyzing a biosample using laser ablation according to any one of claims 1, 2, 3, 4, 5, 6, 7, 8, 9, 10, 11, 12, 13, 14 and 15, wherein
by allowing a tissue image, which is obtained by observing said biosample by a microscope, to correspond to the position of said ablated spot, localization of analytical-target molecules in said biosample is analyzed.

17. The method of analyzing a biosample using laser ablation according to any one of claims 1, 2, 3, 4, 5, 6, 7, 8, 9, 10, 11, 12, 13, 14, 15 and 16, wherein
the pulse time width of said ultra-short pulse laser beams are 1 femto second or more and 1 pico second or less, and the peak value output of the laser beam is 1 mega watt or more and 10 giga watts or less.

18. The method of analyzing a biosample using laser ablation according to any one of claims 1, 2, 3, 4, 5, 6, 7, 8, 9, 10, 11, 12, 13, 14, 15, 16 and 17, wherein
said biosample is a biotissue section or a smear sample.

19. An analyzing apparatus of a biosample using laser ablation, comprising:
an ultra-short pulse laser generating unit capable of outputting ultra-short pulse laser beams, which by irradiating the beams on a biosample to be analyzed and ablating the biosample, atomizes molecules contained in said biosample into constituting elements, and ionizes said atomized constituting elements;
a spectrometer that introduces and analyzes the constituting elements that are ionized by the ultra-short pulse laser beams outputted from said ultra-short pulse laser generating unit; and
a microscope unit for observing the shape of said biosample to be analyzed.

20. The analyzing apparatus of a biosample using laser ablation according to claim 19, wherein
said microscope unit is an upright microscope,
the objective lens of said upright microscope is arranged on the upper surface of said biosample, and the irradiation of the ultra-short pulse laser beams from said ultra-short pulse laser generating unit is performed from the lower surface of said biosample.

21. The analyzing apparatus of a biosample using laser ablation according to claim 19, wherein
said microscope unit is an upright microscope unit,
the objective lens of said upright microscope is arranged on the upper surface of said biosample, and the irradiation of the ultra-short pulse laser beams from said ultra-short pulse laser generating unit is performed from the upper surface of said biosample.

22. The analyzing apparatus of a biosample using laser ablation according to claim 19, wherein
said microscope unit is an inverted microscope,
the objective lens of said inverted microscope is arranged on the lower surface of said biosample, and the irradiation of the ultra-short pulse laser beams from said ultra-short pulse laser generating unit is performed from the upper surface of said biosample.

23. The analyzing apparatus of a biosample using laser ablation according to claim 19, wherein
said microscope unit is an inverted microscope,
the objective lens of said inverted microscope is arranged on the lower surface of said biosample, and the irradiation of the ultra-short pulse laser beams from said ultra-short pulse laser generating unit is performed from the lower surface of said biosample.

24. The analyzing apparatus of a biosample using laser ablation according to any one of claims 19, 20, 21, 22 and 23, wherein
said ultra-short pulse laser generating unit outputs ultra-short pulse laser beams whose pulse time width is 1 femto second or more and 1 pico second or less and whose peak value output is 1 mega watt or more and 10 giga watts or less.

25. The analyzing apparatus of a biosample using laser ablation according to any one of claims 19, 20, 21, 22, 23 and 24, said apparatus further comprising:
an image analysis apparatus that analyzes images observed by said microscope unit.

26. The analyzing apparatus of a biosample using laser ablation according to any one of claims 19, 20, 21, 22, 23, 24 and 25, wherein
said biosample is a biotissue section or a smear sample.
